(19) United States
(12) Patent Application Publication
(10) Pub. No.: US 2021/0353169 A1
(43) Pub. Date: Nov. 18, 2021

(54) DETECTING VASCULAR CONDITIONS IN ANIMAL BODIES

(71) Applicant: ENT. SERVICES DEVELOPMENT CORPORATION LP, Houston, TX (US)

(72) Inventors: Larry SCHMIDT, Rutledge, GA (US); Satwant KAUR, Mountain View, CA (US)

(73) Assignee: ENT. SERVICES DEVELOPMENT CORPORATION LP, Houston, TX (US)

(21) Appl. No.: 17/385,496

(22) Filed: Jul. 26, 2021

Related U.S. Application Data
(62) Division of application No. 15/306,048, filed on Oct. 21, 2016.

(51) Int. Cl. A61B 5/024; A61B 5/021; A61B 5/026; A61B 5/00; A61B 5/0285

(52) U.S. Cl. CPC A61B 5/02444; A61B 5/02125; A61B 5/024; A61B 5/026; A61B 5/002; A61B 2560/0475; A61B 5/0285; A61B 5/6824; A61B 5/0002; A61B 2560/04; A61B 5/0022

(57) ABSTRACT

Examples of 3D-printed sensing devices for detecting vascular conditions in an animal body are described. A 3D-printed sensing device may comprise a binding layer to attach the 3D-printed sensing device to a part of the animal body. A sensor layer is extruded atop the binding layer. The sensor layer comprises a piezoresistive transducer to generate an electrical signal based on a pulse detected in the part of the animal body. In an example, the electrical signal is a binary signal having a logical high value at an instant of occurrence of the pulse and is agnostic of a strength of the pulse. An amplification module in the sensor layer may amplify the electrical signal and provide the amplified signal to a transmitter unit of the 3D-printed sensing device to transmit the amplified signal to a monitoring device associated with the 3D-printed sensing device.

### BACKGROUND

Vascular conditions are disorders relating to circulation of blood in an animal body. Such conditions affect the system of blood vessels which carry blood to different parts of the animal body. Vascular conditions may pose serious health related complications and, thus, are treated, through surgery or medication, as early as they are diagnosed.

Non-invasive techniques may be used for diagnosing an animal body for vascular conditions. Non-invasive methods for detecting vascular conditions involve no skin penetration, no damage to tissues and, therefore, cause no patient discomfort.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the figures to reference like features and components. The following detailed description references the drawings, wherein:

FIG. 1 schematically illustrates a network environment implementing a system for detecting vascular conditions in an animal body, according to an example of the present subject matter;

FIGS. 2A and 2B illustrate a 3D-printed sensing device for detecting vascular conditions in an animal body, according to an example of the present subject matter;

FIG. 3 illustrates a monitoring device for identifying vascular conditions in an animal body, according to an example of the present subject matter;

FIGS. 4A and 4B illustrate example methods for detecting vascular conditions in an animal body; and

FIGS. 5A and 5B illustrate example network environments for manufacturing a 3D-printed sensing device for detecting vascular conditions in an animal body.

### DETAILED DESCRIPTION

Various diagnostic procedures, such as imaging-based tests, enable detection of vascular conditions in an animal body. The repeatability of such tests is usually low and the tests can be performed only periodically. Thus, there exists a possibility of a vascular condition remaining undetected for a significant amount of time, exposing a patient to serious risks. Also, devices for continuous monitoring of a body for vascular conditions that are accurate and simple to use are generally unavailable. Further, manufacturing of the devices used for such medical purposes generally involves procuring and assembling several small components with precision, often making the manufacturing resource and cost intensive.

Aspects of systems and methods for detection of vascular conditions in an animal body are described herein. Further, aspects of the systems and methods for manufacturing a 3D-printed sensing device that provides for detection of vascular conditions in an animal body are also described. In accordance with one example of the present subject matter, the vascular conditions may include, but not limited to, arterial stiffness, blood pressure irregularity, abnormal heart rate, stenosis and blood vessel occlusions such as a blood clot.

For detecting vascular conditions, the 3D-printed sensing device may be attached to any part of the animal body. In an example implementation, the 3D-printed sensing device comprises a piezoresistive transducer which generates an electrical signal based on a pulse detected in a part of the animal body to which the 3D-printed sensing device may have been attached. In accordance with one example implementation, the electrical signal may be a binary signal that may have either a logical high or a logical low value to indicate presence or absence of a pulse at any given time instant. For example, the binary signal may have a logical high value at an instant of occurrence of the pulse and may be agnostic of the strength of the pulse, also known as the pulse pressure or the amplitude of the pulse. The electrical signal may be amplified by an amplification module of the 3D-printed sensing device and transmitted to a monitoring device associated with the 3D-printed sensing device through a transmitter unit of the 3D-printed sensing device.

In an example implementation, the monitoring device may receive a first binary signal indicative of occurrence of a first pulse from a first 3D-printed sensing device placed at a first location on the animal body. Similarly, the monitoring device may also receive a second binary signal indicative of occurrence of a second pulse from a second 3D-printed sensing device placed at a second location on the animal body. It should be noted that the second pulse is caused due to the same systolic motion of the animal heart corresponding to the first pulse sensed at the first location. In an example, the monitoring device computes a time difference in occurrence of the first pulse and the second pulse, and may compare the time difference to a reference time difference value to identify a vascular condition between the first location and the second location.

Further, in some example implementations, the monitoring device may receive several binary signals corresponding to a systolic motion of the animal heart from a plurality of 3D-printed sensing devices, each placed at predetermined locations on the animal body. The monitoring device may compute a blood flow rate in the animal body based on the received binary signals and determine a possible occurrence of a vascular condition in the animal body based on the blood flow rate.

Thus, in some cases, the systems and methods of the present subject matter need not involve use of complex medical devices, and presents a simplified method for detection of vascular conditions. The systems and methods of the present subject matter further provide for continuous monitoring, for example, during a predefined risk period to identifying vascular conditions in animal bodies, such as patients undergoing a surgery and patients under cardiovascular medication.

The 3D-printed sensing device is easy to manufacture and may be printed, for example, by a 3D-printing device as a single unit which may not require any assembling. In an example implementation, the 3D-printed sensing device may include a binding layer of the 3D-printed sensing device for attaching the 3D-sensing device to part of an animal body. Further, a flexible substrate layer may be extruded on the binding layer and a sensor layer may be printed on the substrate layer. In an example, the sensor layer may comprise the above described piezoresistive transducer, amplification module and transmitter unit.

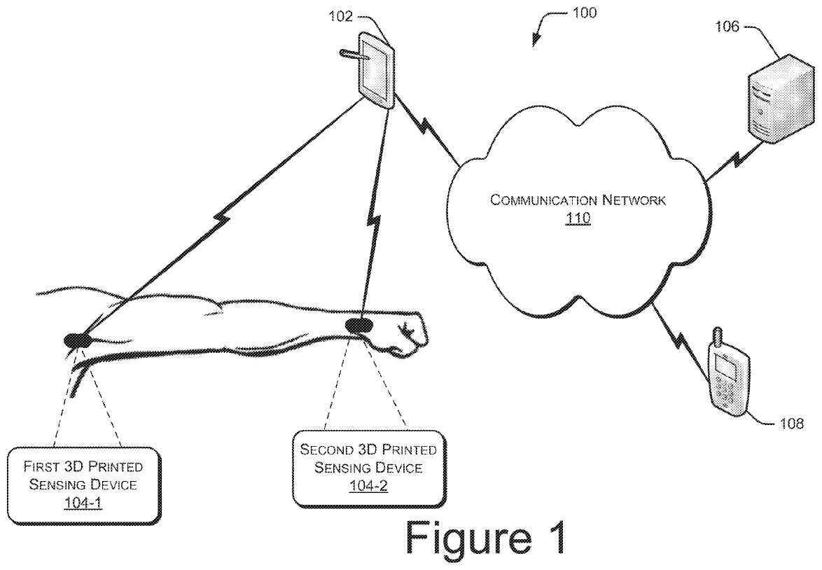

The manner in which the systems and methods for detecting vascular conditions in animal bodies using 3D-printed sensing devices and for manufacturing the 3D-printed sensing devices shall be explained in details with respect to FIGS. 1 to 5. While aspects of described systems and methods can be implemented in any number of different computing systems, environments, and/or configurations, the examples are described in the context of the following example system(s).

FIG. 1 schematically illustrates a network environment 100 implemented to detect vascular conditions in an animal body, interchangeably referred to as body for simplicity, according to an example of the present subject matter. The network environment 100 comprises a monitoring device 102, Examples of the monitoring device 102 may include, but are not limited to, specialized monitoring devices, mobile phones, smart phones, PDAs, tablets, and the like. The monitoring device 102 may be communicatively coupled to a plurality of 3D-printed sensing devices, each attached to a different location of an animal body. Each of the plurality of 3D-printed sensing devices detects an occurrence of a pulse that corresponds to a systolic motion of the animal heart at their respective locations.

As would be understood, each systolic motion of the heart pumps blood into blood vessels that carry the blood to different parts of the body. As blood is propelled through the blood vessels, a tactile throb corresponding to the systolic motion of the heart may be detected at places where the blood vessel is close to the skin. Common places to feel a pulse in a human body include the wrist, neck, temple, behind the knees, and so on.

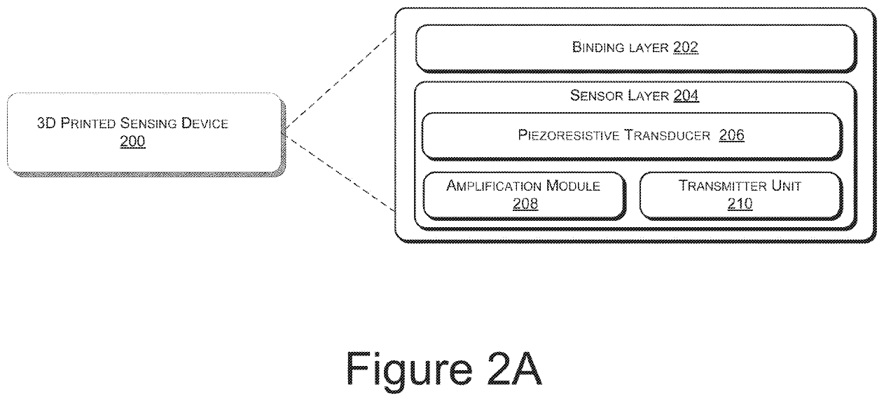

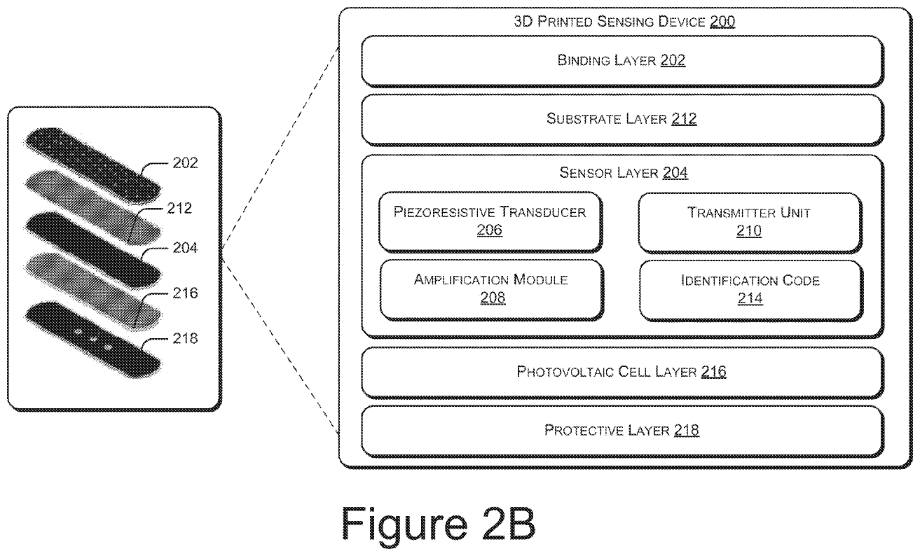

In an example implementation, each of the 3D-printed sensing devices attached to different locations in the animal body produce a binary signal. A binary signal may be understood as a signal pulse having a logical high value at an instant of occurrence of the pulse at respective locations. The implementation and working of the 3D-printed sensing devices of the present subject matter to detect the pulse is explained in details subsequently in conjunction with FIG. 2A and FIG. 2B.

Based on the time difference between occurrences of the pulses, as detected by the 3D-printed sensing devices placed at the different locations, in one example, the monitoring device 102 may compute a blood flow rate in the animal body. The blood flow rate is indicative of a vascular health of the animal body and may be used to identify a possible vascular condition in the animal body. For example, a reduced blood flow rate may be indicative of low blood pressure or reduced heart rate in the animal body while an elevated blood flow rate may indicate high blood pressure or increased heart rate.

In addition to determining the overall vascular health of the animal body, the 3D-printed sensing devices may also be used to detect vascular abnormalities that may occur in localized areas of the animal body. Examples of such localized vascular abnormalities include, but not limited to, arteriosclerosis, stenosis, and atherosclerosis.

Another example of a localized vascular abnormality includes blood clots that may have formed between any two given locations in an animal body, such as a patient with restricted mobility. Detection of formation of blood clots in a localized area of the animal body is explained in reference to an example implementation illustrated in FIG. 1. The figure illustrates a first and a second 3D-printed sensing device 104-1 and 104-2, respectively, from amongst the plurality of 3D-printed sensing devices, attached to the body of the patient, for example, in proximity of an artery or a vein, in the part of the body being monitored for formation of blood clots.

In an example implementation, the first 3D-printed sensing device 104-1 may be placed at a first location on the body of the patient, to detect occurrence of a pulse in the first location. In a similar manner, the second 3D-printed sensing device 104-2 may be placed at a second location to detect the second pulse. The first and the second location may be separated by a predefined distance. Although in the illustrated example implementation, the first 3D-printed sensing device 104-1 is placed in the bicep region while the second 3D-printed sensing device 104-2 is located in the wrist region, the 3D-printed sensing devices may be placed at any other parts of the body for identifying vascular conditions in the respective parts.

The first and the second 3D-printed sensing device 104-1 and 104-2 detect occurrence of a pulse at the respective locations. For example, the first 3D-printed sensing device 104-1 may detect a first pulse at time instant t1 and the second 3D-printed sensing device 104-2 may sense a second pulse at time instant t2. As explained previously, the second pulse corresponds to the same systolic motion of the animal heart that caused the first pulse. The first and the second 3D-printed sensing device 104-1 and 104-2 generate a first and a second binary signal upon detecting the respective pulses. The binary signals may be transmitted to the monitoring device 102.

The monitoring device 102 may receive the first and the second binary signal when the respective pulses occur at different time instant, such as t1 and t2. In the present description, the words during, while, and when are not exact terms that mean an action takes place instantly upon an initiating action but that there may be some small but reasonable delay, such as a propagation delay, between the initial action and the reaction that is initiated by the initial action.

The monitoring device 102 may compute the time difference in occurrence of the first and the second pulse, i.e., the time elapsed between time instant t1 and t2, based on the received first and the second binary signals. Further, the monitoring device 102 compares the computed time difference to a reference time difference value that corresponds to a time difference measured in absence of a blood clot between the first and the second location to identify formation of a blood clot between the two locations.

To explain in reference to the illustrated example, where the first 3D-printed sensing device 104-1 is placed in the bicep region and the second 3D-printed sensing device 104-2 is located in the wrist region, the time difference in occurrence of a first pulse in the bicep region and a second pulse in the wrist region may be computed. The computed time difference may be compared to the reference time difference value that corresponds to a time difference measured in absence of any blood clots between the bicep and the wrist region. If the time difference is higher than the reference time difference value by a predefined threshold, it may indicate that the blood flow rate between the bicep and the wrist region may have decreased, thereby revealing a possibility of formation of blood clots or existence of any other vascular condition in between the bicep and the wrist region.

Once the monitoring device 102 ascertains possible existence of a vascular condition in the animal body, it may generate an alert notification that may alert the patient or a healthcare provider controlling the monitoring device 102 to take corrective measures, such as contacting a physician for suitable medication, further testing, or any further action.

In one example implementation, the alert notification so generated may be communicated to one or more remote communication devices. In some examples, the remote communication device may be a server 106 of a hospital that may maintain health records of patients. In some examples, the remote communication device may be a mobile communication device 108 of a physician or a healthcare provider of a patient.

The monitoring device 102 may communicate with the remote communication devices through a communication network 110. The communication network 110 may be implemented as a wireless network or a wired network, or a combination thereof. The communication network 110 can be an individual network or a collection of many such individual networks, interconnected with each other and functioning as a single large network, e.g., the Internet or an intranet, such as that of a hospital. The communication network 110 can be implemented as one of the different types of networks, such as local area network (LAN), wide area network (WAN), and such. The communication network 110 may either be a dedicated network or a shared network, which represents an association of the different types of networks that use a variety of protocols, for example, Hypertext Transfer Protocol (HTTP), Transmission Control Protocol/Internet Protocol (TCP/IP), etc., to communicate with each other. The communication network 110 may also include individual networks, such as, but are not limited to, GSM network, UMTS network. LTE network, PCS network, TDMA network, CDMA network, NGN, PSTN, and ISDN. The monitoring device 102 and the remote communication devices work on communication protocols that are compatible with the communication network 110 to which the monitoring device 102 and the remote communication devices are coupled.

In accordance with the foregoing description, the systems and methods of the present subject matter provide for continuous monitoring of an animal body, for vascular conditions in any part of the body. For instance, under many circumstances, such as in a post-surgery recovery period, a person's mobility may be limited which increases the likelihood of formation of blood clots in various parts of the body of the person. In such cases, the person may be monitored either continuously or after small intervals of time for an entire duration of a risk period that may be defined for the person based on the condition of the person, for example, by a physician. The monitoring does not require physical examination or in-person visit by a physician. Further, healthcare providers may be made aware of status of vascular heath in patients in hospitals, homes or any other remote locations on an ongoing basis such that corrective measures may be taken promptly.

Further implementation and working of the 3D-printed sensing device is discussed in reference to FIGS. 2A and 2B which illustrate components of a 3D-printed sensing device 200 according to an example of the present subject matter. In accordance with one implementation of the present subject matter, the 3D-printed sensing device 200 may be printed using a 3D-printing device or an additive manufacturing equipment. In other words, the 3D-printed sensing device 200 is manufactured by a process of 3D printing, also known as additive manufacturing, that involves layer-by-layer extrusion of materials that form various components of the 3D-printed sensing device 200.

In accordance with an examples implementation illustrated in FIGS. 2A and 2B, the 3D-printed sensing device 200 comprises a binding layer 202 that enables the 3D-printed sensing device 200 to be attached to a part of an animal body, for example, in the vicinity of a blood vessel where a pulse may be detected. In an example implementation, the binding layer 202 may be made of Polyvinyl Chloride (PVC). Further, in some implementations, the binding layer 202 may comprise a layer of adhesive applied at the surface of the binding layer 202 that contacts with the body to attach the 3D-printed sensing device 200 to the body. In other implementations, the 3D-printed sensing device 200 may also be attached to the body by using adhesive tapes, elastic band or any other similar fastening mechanism that may affix the 3D-printed sensing device 200 to a given location of the body.

The 3D-printed sensing device 200 comprises a sensor layer 204 extruded on top of the binding layer 202. Electronic components that enable detection of a pulse may be included within the sensor layer 204. The electronics components may include, for example, a piezoresistive transducer 206, an amplification module 208 and a transmitter unit 210. In one example implementation, the sensor layer 204 may be extruded atop a substrate layer 212 (shown in FIG. 2B). The substrate layer 212 may be made of a flexible material, such as silicone, and may be provided to act as a base on which the various electronics components of the sensor layer 204 may be extruded. The substrate layer 212 may be flexible, allowing the 3D-printed sensing device 200 to be properly attached to the body.

Suitable materials may be used to print the different electronic components of the sensor layer 204. For example, semiconductor materials, for example, Silicon, Zinc Oxide, Copper Oxide and conductive polymer, such as Poly(3,4-ethylenedioxythiophene (PEDOT:PSS) may be used to print the electronic components. Further, electrically conductive materials, such as silver and copper may be used to extrude connectors between the electronic components of the sensor layer 204.

The piezoresistive transducer 206 may detect a pulse by converting the mechanical energy of the pulse into an electrical signal. This electrical signal may have a low amplitude and may be amplified by the amplification module 208 to enable the transmitter unit 210 to provide the amplified signal to a monitoring device, such as the monitoring device 102 that may be communicatively coupled to the 3D-printed sensing device 200 for further processing of the amplified signal to identify the vascular conditions in the body.

In an example implementation, the electrical signal generated by the piezoresistive transducer 206, i.e., the output of the 3D-printed sensing device 200, may be a binary signal that may have a logical high or a logical low value. In an example the logical low value may be 0 Volts while the logical high value may be any non-zero value, such as 2 Volts, 5 Volts, 8 Volts or any other value depending on the configuration of the amplification module 208. The electrical signal has a logical high value at an instant of occurrence of a pulse in the part of the body to which the 3D-printed sensing device 200 is attached and may be agnostic of the strength of the pulse.

In some example implementations, the 3D-printed sensing device 200 may also detect the strength of a pulse. As mentioned previously, the 3D-printed sensing device 200 detects a tactile throb of a pulse and, as evident, the 3D-printed sensing device 200 may convert the mechanical pressure of the throb is converted into an equivalent electrical signal. Accordingly, in an example, the piezoresistive transducer 206 may generate an electrical signal that has an amplitude proportional to the strength of the pulse being measured. In such implementations, variations in the electrical signal may be analyzed to ascertain possible occurrence of a vascular condition in the animal body. For example, a drop in the amplitude of the electrical signal, for example, more than 40% of a normal amplitude, in a patient who may be under observation, may indicate a possible vascular condition.

The monitoring device 102 operates based on electrical signals received from two or more 3D-printed sensing devices located at different parts of the body separated by a predefined distance to identify vascular conditions in the body. In an example implementation, to enable the monitoring device 102 to determine a location of the 3D-printed sensing device 200, the sensor layer 204 may further include an identification code 214. Reference is made to FIG. 2B illustrating an example implementation of the 3D-printed sensing device 200 for further elaboration.

In an implementation, the identification code 214 may be hardwired in the 3D-printed sensing device 200, for example, by printing the identification code 214 in the sensor layer 204 at the time of manufacturing. In other implementations, a field programmable tag may be printed in the sensor layer 204 during manufacturing of the 3D-printed sensing device 200. Post-manufacturing, the field programmable tag may be configured with the identification code 214, or, in other words, the identification code 214 may be hardcoded in the field programmable tag to store the identification code 214 in the field programmable tag.

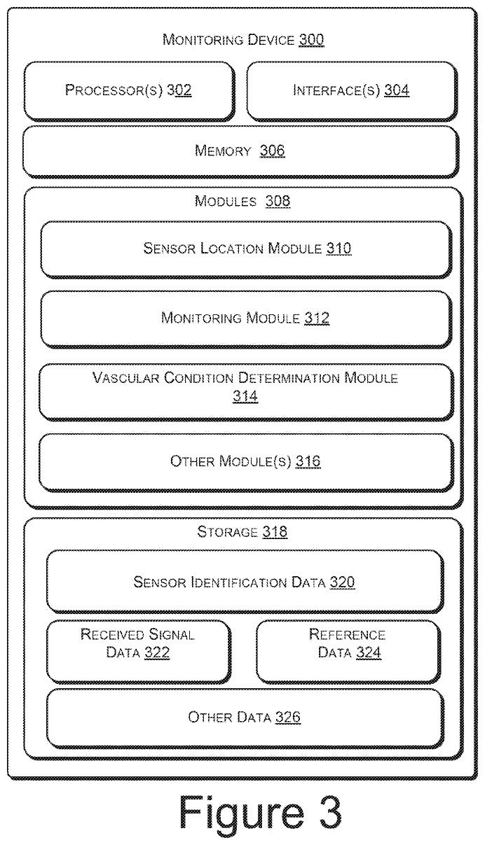

The 3D-printed sensing device 200 may transmit the identification code 214 to the monitoring device 102 through the transmitter unit 210. In an example, the transmitter unit 210 may transmit the identification code 214 periodically. In another example, the identification code 214 may be transmitted prior to sending the electrical signal or after the electrical signal. The monitoring device 102 may process electrical signals received from two or more 3D-printed sensing devices taking into account their corresponding locations on the different parts of the body, to detect vascular conditions on the body. The implementation and working of the monitoring device 102 has been elaborated later in the description of FIG. 3.

In an example implementation, the various electronic components of the 3D-printed sensing device 200, such as the piezoresistive transducer 206, the amplification module 208 and the transmitter unit 210 may derive power from a photovoltaic cell layer 216 of the 3D-printed sensing device 200 for their operation. For example, the photovoltaic cell layer 216 may comprise one or more photovoltaic cells that convert ambient light into electrical energy to power the electronic components of the 3D-printed sensing device 200. For example, a photovoltaic cell may include a cathode layer extruded using a conductive material, such as Indium Tin Oxide: a buffer layer extruded using a conductive polymer material, such as PEDOT:PSS, Poly(3-hexylthiophene-2,5-diyl) (P3HT), Phenyl-C61-butyric acid methyl ester (PCBM); and an anode layer extruded using a conductor, such as Aluminum.

In some example implementations, where the 3D-printed sensing device 200 may not include the photovoltaic cell layer 216, the 3D-printed sensing device 200 may be operated using an external power source. For example, a dry cell, such as a button cell, may be coupled to the 3D-printed sensing device 200 to provide power to the components of the 3D-printed sensing device 200.

Further, in one implementation, a protective layer 218 may be provided over the photovoltaic cell layer 216, or the sensor layer 204 in implementations where the photovoltaic cell layer 216 may not be included in the 3D-printed sensing device 200, to protect the 3D-printed sensing device 200, for example, from dust and moisture. In one implementation, the protective layer 218 may be transparent to allow ambient light to penetrate to the photovoltaic cell layer 216.

Thus, the 3D-printed sensing device 200 may be manufactured as a single unit. This avoids a manufacturing process that involves assembling numerous electronics components together. Further, the 3D-printed sensing device 200 may be a self-powered and self-adhesive device that may be attached to the part of the body to effectively sense pulses occurring in the part of the body based on which the monitoring device 102 may detect vascular conditions in the body.

The implementation and working of a monitoring device to detect vascular conditions in the body is herein explained in reference to FIG. 3 which illustrates a monitoring device 300, according to an example of the present subject matter. Examples of the monitoring device 300 may include, but are not limited to, specialized monitoring devices, mobile phones, smart phones, PDAs, tablets, laptops, and the like.

In one implementation of the present subject matter, the monitoring device 300 includes one or more processor(s) 302. I/O interface(s) 304, and a memory 306 coupled to the processor(s) 302. The processor(s) 302 may be implemented as one or more microprocessors: microcomputers, microcontrollers, digital signal processors, central processing units, state machines, logic circuitries, and/or any devices that manipulate signals based on operational instructions. Among other capabilities, the processor(s) 302 are configured to fetch and execute computer-readable instructions stored in the memory 306.

The functions of the various elements shown in the figure, including any functional blocks labeled as “processor(s)”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), non-volatile storage. Other hardware, conventional and/or custom, may also be included.

The I/O interface(s) 304 may include a variety of software and hardware interfaces, for example, interfaces for peripheral device(s), such as data input output devices, referred to as I/O devices, storage devices, network devices, etc. For example, the monitoring device 300 may interact with the 3D-printed sensing devices through the I/O interface(s) 304. The I/O device(s) may include Universal Serial Bus (USB) ports. Ethernet ports, host bus adaptors, etc., and their corresponding device drivers. The I/O interface(s) 304 facilitate the communication of the monitoring device 300 with various networks, such as the communication network 110 and various remote communication devices, such as the server 106 and the mobile communication device 108.

The memory 306 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

The monitoring device 300 may also include various modules 308. The modules 308, amongst other things, include routines, programs, objects, components, data structures, etc., which perform particular tasks or implement particular abstract data types. The modules 308 may also be implemented as, signal processor(s), state machine(s), logic circuitries, and/or any other device or component that manipulate signals based on operational instructions.

Further, the modules 308 can be implemented in hardware, instructions executed by a processing unit, or by a combination thereof. The processing unit can comprise a computer, a processor, such as the processor 302, a state machine, a logic array or any other suitable devices capable of processing instructions. The processing unit can be a general-purpose processor which executes instructions to cause the general-purpose processor to perform the required tasks or, the processing unit can be dedicated to perform the required functions.

In another aspect of the present subject matter, the modules 308 may be machine-readable instructions (software) which, when executed by a processor/processing unit, perform any of the described functionalities. The machine-readable instructions may be stored on an electronic memory device, hard disk, optical disk or other machine-readable storage medium or non-transitory medium. In one implementation, the machine-readable instructions can be also be downloaded to the storage medium via a network connection.

The module(s) 308 further include a sensor location module 310, a monitoring module 312, a vascular condition determination module 314, and other module(s) 316. The other module(s) 316 may include programs or coded instructions that supplement applications and functions of the monitoring device 300.

The monitoring device 300 may further include storage 318, which amongst other things, serves as a repository for storing data processed, received, associated, and generated by one or more of the module(s) 308. Storage 318 includes, for example, sensor identification data 320, received signal data 322, reference data 324 and other data 326. The other data 326 includes data generated as a result of the execution of one or more modules in the other module(s) 316.

As described previously, the 3D-printed sensing devices associated with the monitoring device 300 may transmit their respective identification codes to the monitoring device 300. The sensor location module 310 of the monitoring device 300 may receive these identification codes. In some example implementations, each of the identification codes may have ‘n’ bits, where ‘n’, i.e., the number of bits depends on the number of 3D-printed sensing devices placed at different locations of a body. In an example where eight 3D-printed sensing devices may be placed on a body, each of the eight 3D-printed sensing devices may be associated with a 3 bit code, such as 000, 001, 010, 011 and so on, that the respective 3D-printed sensing devices may transmit to the monitoring device 300.

In the monitoring device 300, each of the identification codes may be mapped to a location of the respective 3D-printed sensing device to which the identification code corresponds. For example, a user may provide inputs to the monitoring device 300 to map the identification codes 000, 001, and 010 to heart, bicep of the right arm and wrist of the right arm, respectively. This data may be stored as sensor identification data 320 in the monitoring device 300. Accordingly, when an identification code is received by the sensor location module 310, the location of the 3D-printed sensing device sending the identification code may be determined based on the sensor identification data 320.

In addition, the monitoring device 300 may also store a predetermined distance between each of the locations. Referring again to the previous example, the user may provide inputs to store the distance between the heart and the bicep of the right arm, the distance between the heart and the wrist of the right arm and the distance between the bicep and the wrist of the right arm in the monitoring device 300. In one example, the inputs may be provided by measuring the distance between the locations of the 3D-printed sensing devices placed on a given body under observation. In other example, the inputs may be based on a predefined standard distances defined for animal bodies of different types based on factors like the height and weight of the body.

As mentioned previously, each of the 3D-printed sensing devices associated with monitoring device 300 provides a binary signal to the monitoring device 300 to indicate a time instant of occurrence of a pulse in their respective locations. The monitoring module 312 of the monitoring device 300 may receive the binary signals. In an example implementation, the each binary signal may be preceded by the identification code of the 3D-printed sensing device generating the binary signal. The monitoring module 312 may store the received binary signals and identification codes as received signal data 322.

In an example implementation, the vascular condition determination module 314 may determine the blood flow rate in the body based on the time difference in occurrence of the pulses, as indicated by the binary signals received from the 3D printed sensing devices, and the predetermined distance between the 3D printed sensing devices. The blood flow rate provides an indication of the vascular health of the body and enables identification of possible vascular conditions. Vascular conditions, such as abnormalities in blood pressure and pulse rate that effect an animal body in general as well as localized vascular conditions, such as arteriosclerosis, atherosclerosis and blood vessel occlusions, such as blood clots that may occur in localized areas in the body, may be identified in accordance with the methods and systems of the present subject matter.

In operation, the monitoring module 312 may receive a first binary signal from a 3D-printed sensing device that senses a first pulse at time instant t1. In one example, the binary signal is received along with the identification code associated with the 3D-printed sensing device. Similarly, a second binary signal and identification code may be received from another sensing device that senses a second pulse, corresponding to the same systolic motion that caused the first pulse, at time instant t2. The monitoring module 312 computes the time difference between time instant t1 and t2 and also identifies the location of the two 3D-printed sensing devices based on the respective identification codes to determine the distance between the two 3D-printed sensing devices. Based on the distance, the vascular condition determination module 314 may compare the computed time difference to a reference time difference value to identify a vascular condition between the location of the two 3D-printed sensing devices. In one example, the reference time difference value may be stored as reference data 324 in the monitoring device 300.

To explain, reference is made to the example considered in the foregoing description where three 3D-printed sensing devices associated with identification codes 000, 001, and 010, respectively, are placed at the heart, bicep of the right arm, and wrist of the right arm, respectively. In an example, the 3D-printed sensing device placed at the heart may send a binary signal indicative of occurrence of a pulse at the heart along with identification code 000 to the monitoring module 312. The pulse occurs as the heart pumps blood into an artery. As the blood flows along the artery and reaches the bicep, the 3D-printed sensing device placed at the bicep may detect the pulse and may send a binary signal along with identification code 001 to the monitoring module 312. The monitoring module 312 computes the time elapsed between the receipt of the two binary signals to determine the time difference in occurrence of the pulse at the heart and the bicep.

Further, the monitoring module 312 determines the distance between the two 3D-printed sensing devices, for example, based on the inputs stored by the user. In the present example, the distance may be considered to be 0.3 meters. A reference time difference value for blood to travel 0.3 meters in a body is referred to determine if the elapsed time deviates from the reference time difference by a predefined threshold. For example, if the time elapsed is more compared to the reference time difference value, it may indicate a reduced blood flow rate between the heart and the bicep, thereby indicating existence of a localized vascular condition, such as narrowing of an artery or clotting of blood between the two locations.

In one implementation, abnormality in the blood flow rate between any two locations, such as the heart and the bicep, for example, may also be indicative of existence of a vascular condition, such as an abnormal blood pressure that may be persisting in the body and may not be localized in any area of the body.

In an example implementation, the reference time difference value may be a normal time difference value measured in absence of a vascular condition in the body. In an example situation where a patient undergoing a surgery is to be monitored for thrombosis in a post-surgery recovery period, readings of normal time difference value may be measured prior to the surgery when no blood clots are present in the body of the patient. Readings of normal time difference values corresponding to different locations where 3D-printed sensing devices may be placed for monitoring formation of blood clots may be recorded and stored in the reference data 324.

In an example implementation, the reference time difference value may be a standard time difference value which may be a predefined time taken for blood to flow a given distance in healthy animal bodies. The standard time difference values corresponding to different distances may be obtained from an external source and stored in the reference data 324 as the reference time difference value. Accordingly, comparison of the time difference measured in a body being monitored and the reference time difference value may allow identification of vascular conditions.

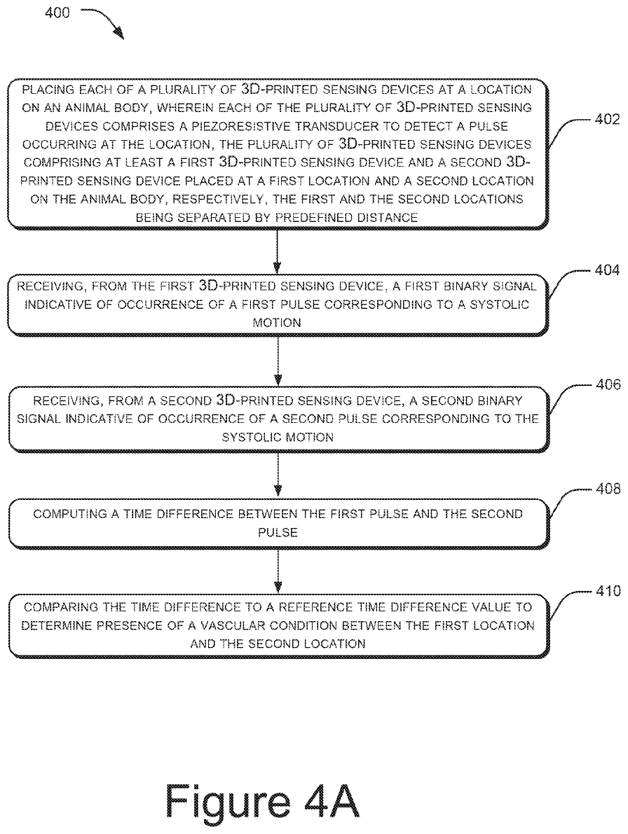

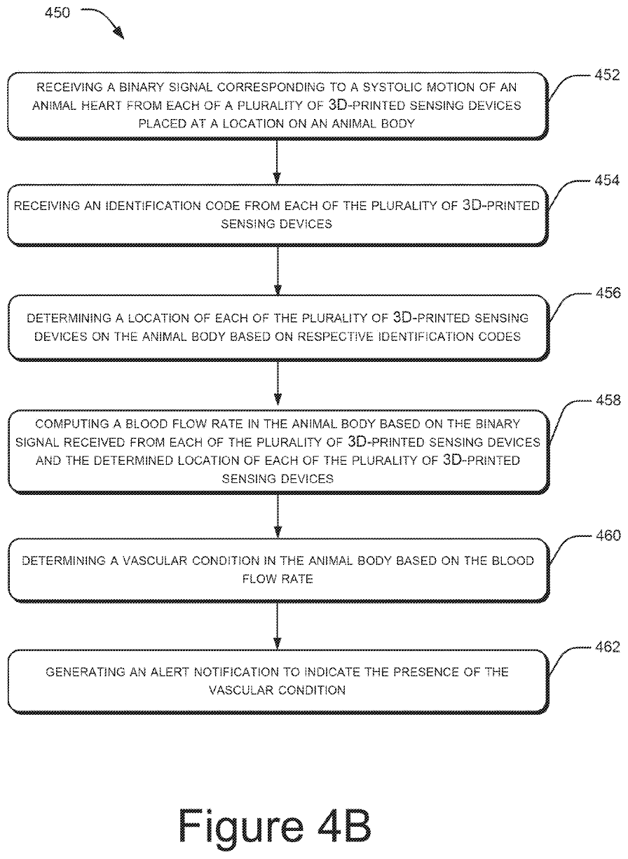

FIGS. 4A and 4B illustrate example methods 400 and 450 for detecting vascular conditions within an animal body. The order in which the methods 400 and 450 are described is not intended to be construed as a limitation, and any number of the described method blocks can be combined in any order to implement the methods 400 and 450, or alternative methods. Additionally, individual blocks may be deleted from the methods 400 and 450 without departing from the spirit and scope of the subject matter described herein. Furthermore, the methods 400 and 450 can be implemented in any suitable hardware, software, firmware, or combination thereof.

A person skilled in the art will readily recognize that steps of the methods 400 and 450 can be performed by programmed computing devices. Herein, some examples are also intended to cover program storage devices, for example, digital data storage media, which are machine or computer readable and encode machine-executable or computer-executable programs of instructions, wherein said instructions perform some or all of the steps of the described method. The program storage devices may be, for example, digital memories, magnetic storage media, such as a magnetic disks and magnetic tapes, hard drives, or optically readable digital data storage media. The examples are also intended to cover both communication network and communication system configured to perform said steps of the example method.

Further, although the methods 400 and 450 for detecting vascular conditions may be implemented in a variety of computing devices, communication devices and specialized monitoring devices, in examples described in FIGS. 4A and 4B, the methods 400 and 450 are explained in context of the aforementioned monitoring device 300 for the ease of understanding.

Referring, to FIG. 4A, at block 402, a plurality of 3D-printed sensing devices are placed at different locations on the animal body. Each of the plurality of 3D-printed sensing devices comprises a piezoresistive transducer to detect a pulse occurring at their respective locations. The plurality of 3D-printed sensing devices comprise at least a first 3D-printed sensing device and a second 3D-printed sensing device placed at a first location and a second location on the animal body, respectively. The first and the second locations may be separated by predefined distance.

At block 404, a first binary signal indicative of occurrence of a first pulse at the first location is received from the first 3D-printed sensing device. Similarly, at block 406, a second binary signal indicative of occurrence of a second pulse, at the second location on the body is received from the second 3D-printed sensing device. As mentioned previously, the second pulse may be caused due to the same systolic motion of the animal heart corresponding to the first pulse sensed at the first location.

As explained previously, the first and the second 3D-printed sensing devices may generate the respective binary signals when the pulses occur at the first and the second location. The binary signals have a logical high value at an instant of occurrence of the pulses and may not vary based on the strength, i.e., amplitude of the detected pulse.

Based on the first and the second binary signals received at blocks 404 and 406, at block 408, a time difference between the first pulse and the second pulse may be computed. At block 410, the computed difference may be compared to a reference time difference value to identify a vascular condition between the first location and the second location. The reference time difference value may be based on the predefined distance that separates the first and the second locations as described previously.

The method 400 as described above may be implemented in a monitoring device, such as the monitoring device 300 for continuous monitoring for vascular conditions in animal bodies. While binary signals received from the first and second 3D-printed sensing devices enable identification of a vascular condition in a localized area between the two devices, a plurality of binary signals from different 3D-printed sensing devices, each placed at a predetermined part of the body, may enable identification of other vascular conditions in the animal body that may not be localized in an area of the body, as explained with reference to method 450 illustrated in FIG. 4B.

At block 452, a binary signal corresponding to a systolic motion of an animal heart is received from each of a plurality of 3D-printed sensing devices that may be placed at different locations on an animal body. As explained previously, each binary signal may be preceded or followed by an identification code of the 3D-printed sensing device generating the binary signal. Accordingly, at block 454, an identification code may be received from each of the plurality of 3D-printed sensing devices. At block 456, the respective locations of each of the 3D-printed sensing devices may be determined based on the received identification codes.

Based on the binary signal received from each of the 3D-printed sensing devices and the determined location of each of the 3D-printed sensing devices, at block 458, a blood flow rate in the animal body is computed. As would be understood, any given 3D-printed sensing device, located at a part of the body, is separated from other 3D-printed sensing devices, located at other parts of the body, by a predefined distance. The predefined distance between the given 3D-printed sensing device and any of the other 3D-printed sensing devices may be determined based on their respective locations identified at block 456. In an example implementation, the 3D-printed sensing devices may be considered in pairs. The blood flow rate may be calculated based on the predefined distance between a pair of 3D-printed sensing devices and the time difference between the respective binary signals received from the pair of 3D-printed sensing devices. In one example, the blood flow rate in the animal body may be an average of the blood flow rate obtained from several pairs of the 3D-printed sensing devices.

At block 460, a vascular condition in the animal body may be determined based on the blood flow rate. The blood flow rate is indicative of the vascular health of an animal body and enables identification of vascular conditions, such as abnormalities in blood pressure and pulse rate. In case existence of a vascular condition in the animal body is ascertained, an alert notification may be generated at block 462. For example, the alert notification may be transmitted to a healthcare provider of the patient to take various measures, such as contacting a physician.

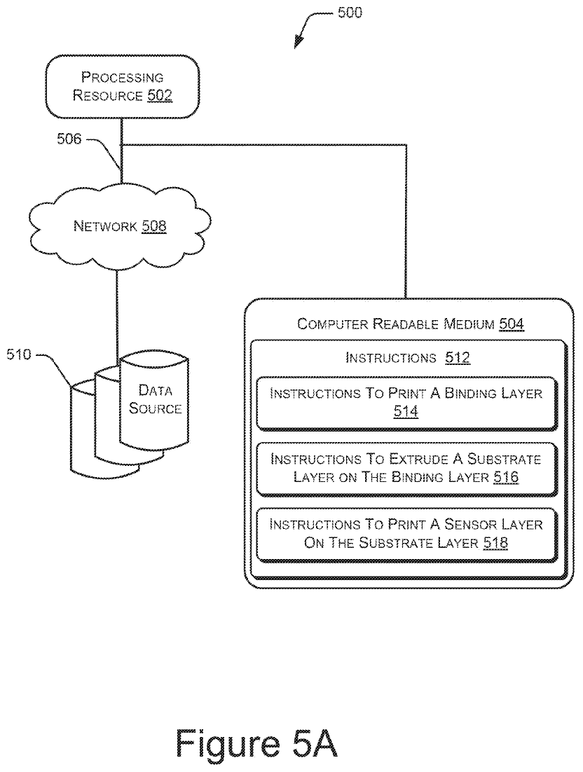

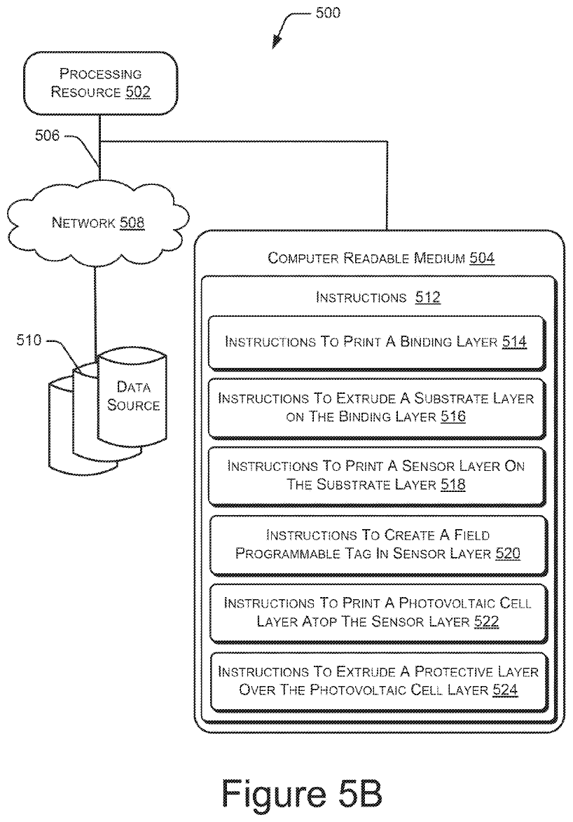

FIGS. 5A and 5B illustrate example network environments 500 implementing a non-transitory computer readable medium for manufacturing a 3D-printed sensing device, such as the 3D-printed sensing device 200 in accordance with an example of the present subject matter. The network environment 500 may be a public networking environment or a private networking environment. In one implementation, the network environment 500 includes a processing resource 502 communicatively coupled to a non-transitory computer readable medium 504 through a communication link 506.

For example, the processing resource 502 can be a processor of a 3D-printing device. The non-transitory computer readable medium 504 can be, for example, an internal memory device or an external memory device. In one implementation, the communication link 506 may be a direct communication link, such as one formed through a memory read/write interface, in another implementation, the communication link 506 may be an indirect communication link, such as one formed through a network interface. In such a case, the processing resource 502 can access the non-transitory computer readable medium 504 through a network 508. The network 508 may be a single network or a combination of multiple networks and may use a variety of different communication protocols.

The processing resource 502 and the non-transitory computer readable medium 504 may also be communicatively coupled to data sources 510 over the network 508. The data sources 510 can include, for example, databases and computing devices. The data sources 510 may be used by a user of the 3D-printing device to communicate with the processing resource 502, for example, to print the 3D-printed sensing device 200.

In one implementation, the non-transitory computer readable medium 504 may include a set of computer readable instructions for printing the 3D-printed sensing device 200. The set of computer readable instructions, referred to as instructions 512 hereinafter, can be accessed by the processing resource 502 through the communication link 506 and subsequently executed to perform acts for printing the 3D-printed sensing device 200.

For discussion purposes, the execution of the instructions 512 by the processing resource 502 has been described with reference to various components introduced earlier with reference to description of FIGS. 2A and 26.

Referring to FIG. 5A, in an example, the instructions 512 include instructions 514 to cause the processing resource 502 to print a binding layer 202 of the 3D-printed sensing device 200. The binding layer 202 may be attached to an animal body, that is to be monitored for vascular conditions, for example, using adhesive. In one example, the lowermost layer of the binding layer that comes in contact with the body may be printed as an adhesive layer.

Instructions 512 may further include instructions 516 that may cause the processing resource 502 may to extrude a flexible substrate layer 212 on the binding layer 202 and instructions 518 that may cause the processing resource 502 to extrude a sensor layer 204 atop the substrate layer 212. In one implementation, the instructions 518 may cause the processing resource 502 to print the components of the sensor layer 204, The sensor layer 204 comprises a piezoresistive transducer 206 to generate an electrical signal upon detecting a pulse in the part of the body where the 3D-printed sensing device 200 may be attached to. Further the sensor layer 204 may also comprise an amplification module 208 to amplify the electrical signal and a transmitter unit 210 to transmit the amplified signal to a monitoring device, such as the monitoring device 300 communicatively coupled to the 3D-printed sensing device 200.

In accordance with an example implementation, the instructions 512 may further comprise instructions 520 that may cause the processing resource 502 to hardwire an identification code 214 in the sensor layer 204. In some implementations, as illustrated in FIG. 5B, the instructions 520 may cause the processing resource 502 to create a field programmable tag in the sensor layer 204 such that the identification code 214 may be stored in the field programmable tag.

Further, in one example implementation, the processing resource 502 may execute instructions 522 to print a photovoltaic cell layer 216 atop the sensor layer 204. The photovoltaic cell layer 216 comprises photovoltaic cells that provide power to the components of the sensor layer 204 for their operation. In addition, the instructions 512 may also include instructions 524, which upon being executed by the processing resource 502 cause a protective layer 218 to be extruded over the photovoltaic cell layer 216. The protective layer 218 may be extruded over the photovoltaic cell layer 216 to protect the 3D-printed sensing device 200 from dust and moisture that may hamper the working of the components of the 3D-printed sensing device 200.

The 3D-printed sensing device 200 manufactured using the 3D-printed in accordance with the process as described above is cost effective and simple to use. The 3D-printed sensing device 200 generates binary signals that are further processed by the monitoring device 300 for identifying vascular conditions in the body.

Thus, the methods and systems of the present subject matter provide for identifying vascular conditions in animal bodies in a continuous and error-free manner. Additionally, the methods and systems of the present subject matter provide for manufacturing of a 3D-printed sensing device.

Although implementations for the 3D-printed sensing devices and monitoring devices have been described in language specific to structural features and/or methods, it is to be understood that the appended claims are not necessarily limited to the specific features or methods described. Rather, the specific features and methods are disclosed as example implementations for detecting vascular conditions in animal bodies and for manufacturing 3D-printed sensing devices that enable detection of vascular conditions in animal bodies.

1-11. (canceled)

12. A non-transitory computer-readable medium storing instructions for printing a 3D-printed sensing device, the instructions being executable by a processing resource of a 3D-printer to cause the 3D-printer to perform operations, comprising:
print a binding layer to attach to a part of an animal body;
extrude a flexible substrate layer on the binding layer; and
print a sensor layer on the substrate layer, wherein the sensor layer comprises:
a piezoresistive transducer to generate an electrical signal on detecting a pulse in the part of the animal body;
an amplification module to amplify the electrical signal; and
a transmitter to transmit the amplified signal to a monitoring device associated with the 3D-printed sensing device.

13. The non-transitory computer-readable medium recited in claim 12, the operations further comprising print a photovoltaic cell layer atop the sensor layer.

14. The non-transitory computer-readable medium recited in claim 12, the operations further comprising create a field programmable tag in the sensor layer, wherein the field programmable tag stores an identification code.

15. The non-transitory computer-readable medium recited in claim 13, the operations further comprising extrude a protective layer over the photovoltaic cell layer.

16. A 3D-printer, comprising:
a non-transitory computer-readable medium storing instructions; and
a processing resource programmed to cooperate with the instructions to cause the 3D-printer to perform operations to print a 3D-printed sensing device, the operations comprising:
print a binding layer to attach to a part of an animal body;
extrude a flexible substrate layer on the binding layer; and
print a sensor layer on the substrate layer, wherein the sensor layer comprises:
a piezoresistive transducer to generate an electrical signal on detecting a pulse in the part of the animal body;
an amplification module to amplify the electrical signal; and
a transmitter to transmit the amplified signal to a monitoring device associated with the 3D-printed sensing device.

17. The 3D-printer recited in claim 16, the operations further comprising print a photovoltaic cell layer atop the sensor layer.

18. The 3D-printer recited in claim 16, the operations further comprising create a field programmable tag in the sensor layer, wherein the field programmable tag stores an identification code.

19. The 3D-printer recited in claim 17, the operations further comprising extrude a protective layer over the photovoltaic cell layer.

20. A method executable by a 3D-printer to print a 3D-printed sensing device, the method comprising:
printing a binding layer to attach to a part of an animal body;
extruding a flexible substrate layer on the binding layer; and
printing a sensor layer on the substrate layer, wherein the sensor layer comprises:
a piezoresistive transducer to generate an electrical signal on detecting a pulse in the part of the animal body;
an amplification module to amplify the electrical signal; and
a transmitter to transmit the amplified signal to a monitoring device associated with the 3D-printed sensing device.

21. The method recited in claim 20, further comprising printing a photovoltaic cell layer atop the sensor layer.

22. The method recited in claim 20, further comprising create a field programmable tag in the sensor layer, wherein the field programmable tag stores an identification code.

23. The method recited in claim 21, further comprising extrude a protective layer over the photovoltaic cell layer.
